(19) United States
(12) Patent Application Publication
(10) Pub. No.: US 2019/0366844 A1
(43) Pub. Date: Dec. 5, 2019

(54) METHOD, SYSTEM, AND VEHICLE FOR PREVENTING DROWSY DRIVING

(71) Applicant: LG ELECTRONICS INC., Seoul (KR)

(72) Inventors: Hee Nam YOON, Seoul (KR); Tae Hwan KIM, Seoul (KR); Seung Bum HONG, Seoul (KR); Beom Oh KIM, Gyeonggi-do (KR)

(73) Assignee: LG ELECTRONICS INC., Seoul (KR)

(21) Appl. No.: 16/543,030

(22) Filed: Aug. 16, 2019

(30) Foreign Application Priority Data
Jul. 26, 2019 (KR) 10-2019-0091060

(51) Int. Cl. B60K 28/06; B60W 40/09; B60W 50/16; A61B 5/18; A61B 5/00; B60W 10/04; B60W 10/20

(52) U.S. Cl. CPC B60K 28/06; B60W 40/09; B60W 50/16; B60W 2040/0827; A61B 5/6893; B60W 10/04; B60W 10/20; A61B 5/18

(57) ABSTRACT

A method for preventing drowsy driving in consideration of an alertness of the driver is disclosed. A method for preventing drowsy driving according to an exemplary embodiment of the present disclosure includes determining an alertness level of a driver based on monitoring information from at least one monitoring unit equipped in a vehicle, determining a stimulus corresponding to at least one of a plurality of stimulation units, based on the determined alertness level, and operating at least one stimulation unit corresponding to the stimulus. A stimulus to be delivered to the driver is determined by a machine learning or deep learning technology using an artificial neural network which has been trained to output a stimulus suitable to improve an alertness of the driver depending on the identity and the state of the driver. According to the present disclosure, an optimal stimulus in accordance with the identity of the driver and the state of the driver is delivered to the driver so that a drowsy driving prevention effect can be improved.

### CROSS-REFERENCE TO RELATED APPLICATION

Pursuant to 35 U.S.C. § 119(a), this application claims the benefit of earlier filing date and right of priority to Korean Patent Application No. 10-2019-0091060, filed on Jul. 26, 2019, the contents of which are hereby incorporated by reference herein in its entirety.

### BACKGROUND

### 1. Technical Field

The present disclosure relates to a vehicle, and more particularly, to an in-car system for preventing drowsy driving and a control method thereof.

### 2. Description of the Related Art

A car accident caused by drowsy driving is highly likely to lead to a big accident because a brake operation is not performed or the brake operation is performed too late. For this reason, studies have shown that the risk of the drowsy driving is not less serious than the risk of drunken driving. In the automotive manufacturing field, efforts have been made to detect and prevent the drowsy driving.

Korean Patent Application Publication No. 10-2014-0007444 (Patent Document 1) discloses a method of determining whether a driver dozes off and modifying control of one or more vehicle systems when the user is dozing off.

Korean Patent Application Publication No. 10-2019-0042259 (Patent Document 2) discloses a method of detecting states of a driver and a vehicle and providing a cognitive load to the driver when the number of factors which affect the driving of the vehicle exceeds a predetermined threshold.

Even though Patent Documents 1 and 2 disclose providing an auditory or tactile stimulus to the driver to prevent the drowsy driving, an algorithm for selecting a stimulus to be provided to the user among a plurality of stimuli is not disclosed. In Patent Documents 1 and 2, it is unclear whether the arbitrarily selected stimulus is effective to improve the alertness of the driver.

### SUMMARY OF THE INVENTION

The inventors of the present invention found out that even though the same stimulus is applied to different drivers, an alertness effect of the stimulus may vary depending on the drivers.

Further, the inventors of the present invention found out that even though the same stimulus is applied to the same driver, an alertness effect of the stimulus may vary depending on an alertness level of the driver.

An object to be achieved by the present disclosure is to provide a stimulus effective to improve the alertness of a driver in a driver-specific manner.

Another object to be achieved by the present disclosure is to provide a stimulus effective to improve the alertness of a driver depending on the state of the driver.

Still another object to be achieved by the present disclosure is to evolve a drowsy driving preventing system to determine an optimal stimulus by learning a result of improving the alertness of the driver by the delivery of the stimulus.

The object of the present disclosure is not limited to the above-mentioned objects and other objects and advantages of the present disclosure which have not been mentioned above may be understood by the following description and become more apparent from exemplary embodiments of the present disclosure. Further, it is understood that the objects and advantages of the present disclosure may be embodied by the means and a combination thereof in the claims.

A method and a vehicle for preventing drowsy driving according to the exemplary embodiment of the present disclosure monitor a state of a driver to determine an alertness level of the driver, select a stimulus to be delivered to the driver based on the determined alertness level and deliver the stimulus to the driver.

According to a first aspect of the present disclosure, a method for preventing drowsy driving comprises: determining a first alertness level of a driver based on monitoring information from at least one monitoring unit equipped in a vehicle; determining a first stimulus corresponding to at least one of a plurality of available stimulation units, based on the determined first alertness level; and operating at least one stimulation unit corresponding to the first stimulus.

According to a second aspect of the present disclosure, a vehicle comprises: at least one monitoring unit to monitor a state of a driver; a plurality of stimulation units to deliver a stimulus to the driver; and a control device. In this case, the control device is configured to determine a first alertness level of a driver based on monitoring information from at least one monitoring unit, determine a first stimulus corresponding to at least one of the plurality of stimulation units, based on the determined first alertness level; and output a control signal to operate a first stimulation unit corresponding to the first stimulus.

According to an exemplary embodiment, the first stimulus may be determined based on an identified identity of the driver and the first alertness level.

According to another exemplary embodiment, when a variation of an alertness level of the driver after delivering the stimulus is equal to or lower than a first threshold value, a second stimulus which is of a different type from the first stimulus may be delivered to the driver. By doing this, a stimulus having a less performance can be excluded.

According to another exemplary embodiment, when a variation of an alertness level of the driver after delivering the stimulus exceeds the first threshold value, but is equal to or lower than a second threshold value, the first stimulus with a changed property may be delivered to the driver. By doing this, the effect of the stimulus which already shows a positive performance can be further improved.

According to another exemplary embodiment, an interactive conversation may be determined as the first stimulus and when a variation of the post-stimulation alertness level exceeds the first threshold value, but is equal to or lower than a second threshold value, a subject of the interactive conversation may be changed.

According to another exemplary embodiment, the first stimulus may be determined based on output data from an artificial neural network having a first alertness level of the driver as input data and the artificial neural network may be trained by an alertness level of the driver or the variation of the alertness level after delivering the stimulus. By doing this, the system of the vehicle can evolve to output an optimal stimulus in accordance with the identity and the state of the driver.

According to the present disclosure, a stimulus to be delivered to the driver is determined based on an identity and an alertness level of the driver so that a stimulus effective to prevent the drowsy driving can be provided in a driver-specific manner and in accordance with the state of the driver.

Further, according to the present disclosure, a type or a property of the stimulus is changed based on a variation of an alertness level of the driver after delivering the stimulus so that a stimulus in which a positive performance is insufficient is excluded or an effect of a stimulus which shows a positive performance can be further improved.

Further, according to the present disclosure, a stimulus to be delivered to the driver is determined using an artificial neural network and the artificial neural network is trained with the performance obtained by the stimulus as training data to evolve a system of a vehicle to output an optimal stimulus in accordance with the identify and the state of the driver.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 is an exemplary diagram of an internal environment of a vehicle according to an exemplary embodiment of the present disclosure;

FIG. 2 is a block diagram schematically illustrating an in-car system for preventing the drowsy driving according to an exemplary embodiment of the present disclosure;

FIG. 3 is a flowchart illustrating an exemplary method of improving an alertness of a driver according to an exemplary embodiment of the present disclosure;

FIGS. 4A to 4C illustrate exemplary graphs showing a change in an alertness of a driver over time after applying a stimulus;

FIG. 5 is a flowchart illustrating an exemplary method of improving an alertness of a driver according to another exemplary embodiment of the present disclosure; and

FIG. 6 is a view illustrating an exemplary scenario for improving an alertness of a driver according to an exemplary embodiment of the present disclosure.

### DETAILED DESCRIPTION

Advantages and features of the present disclosure and methods of achieving the advantages and features will be more apparent with reference to the following detailed description of example embodiments in connection with the accompanying drawings. However, the description of particular example embodiments is not intended to limit the present disclosure to the particular example embodiments disclosed herein, but on the contrary, it should be understood that the present disclosure is to cover all modifications, equivalents and alternatives falling within the spirit and scope of the present disclosure. The example embodiments disclosed below are provided so that the present disclosure will be thorough and complete, and also to provide a more complete understanding of the scope of the present disclosure to those of ordinary skill in the art. In the interest of clarity, not all details of the relevant art are described in detail in the present specification in so much as such details are not necessary to obtain a complete understanding of the present disclosure.

The terminology used herein is used for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “includes,” “including,” “containing,” “has,” “having” or other variations thereof are inclusive and therefore specify the presence of stated features, integers, steps, operations, elements, and/or components, but do not preclude the presence or addition of one or more other features, integers, steps, operations, elements, components, and/or groups thereof. The terms such as “first,” “second,” and other numerical terms may be used herein only to describe various elements and only to distinguish one element from another element, and as such, these elements should not be limited by these terms.

Hereinbelow, the example embodiments of the present disclosure will be described in greater detail with reference to the accompanying drawings, and on all these accompanying drawings, the identical or analogous elements are designated by the same reference numeral, and repeated description of the common elements will be omitted.

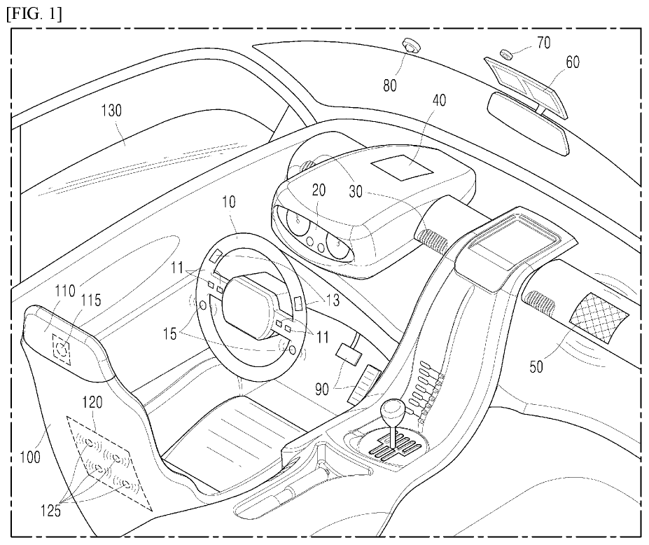

FIG. 1 is an exemplary diagram of an internal environment of a vehicle according to an exemplary embodiment of the present disclosure.

Referring to FIG. 1, a steering wheel 10, an instrument panel 20, an air conditioner 30, a display screen 40, a speaker 50, an interior light 60, an acoustic sensor 70, and an image sensor 80 may be disposed in front of a driver's seat.

The steering wheel 10 may include control buttons 11 which are capable of setting configurations of the vehicle. For example, the control buttons 11 may be used to control the instrument panel 20 or display of the display screen 40 or control a volume of the speaker 50. Further, the control buttons 11 may be used to set speed control (cruise control) or select one of a plurality of driver profiles stored in a memory.

Further, the steering wheel 10 may include a biometric sensor 13 which detects biometric information of the driver. The biometric sensor 13 may be a heart rate and/or oxygen saturation sensor located in a place where the driver's thumb or index finger is disposed.

Further, the steering wheel 10 may include a vibration unit 15 which delivers a vibration to the driver as the stimulus. For example, the vibration unit 15 may be located in a place where a palm of the driver is disposed.

The instrument panel 20 may include, for example, a gauge which displays a driving speed of the vehicle, a revolution per minute (RPM) of an engine, and an amount of fuel.

The air conditioner 30 may provide flow of air at a fan speed and a temperature set by a user or a control device. For example, the air conditioner 30 may discharge air which is heated or cooled at a predetermined temperature at a predetermined fan speed.

The display screen 40 may be any type of device which is capable of providing various additional information (for example, navigation information) to the driver, in addition to information provided by the instrument panel 20, and for example, may be a head-up display (HUD).

The speaker 50 may convert an electrical signal into sound to output the sound. For example, the speaker 50 may output music from a radio receiver, a CD player, or an MP3 player or output an audio guidance from a navigation, or music, a voice, or a warning sound in accordance with a control signal from a control device of the vehicle.

The interior light 60 illuminates the internal environment of the vehicle or emits light directed to the driver in accordance with a manual setting of the user, an open/closed state of the door, or a control signal from the control device.

The acoustic sensor 70 is configured to acquire sounds in the vehicle, specifically, a voice of the driver, and for example, may be a microphone. The acoustic sensor 70 may be mounted in any location appropriate to acquire the voice of the driver. For example, the acoustic sensor 70 may be mounted on an indoor ceiling of the vehicle or combined in a module of the interior light 60.

The image sensor 80 is configured to acquire images in the vehicle, specifically, an image of the driver, and for example, may be a camera. The image sensor 80 may be mounted in any location appropriate to acquire the image of the driver. For example, the image sensor 80 may be mounted on the indoor ceiling of the vehicle, mounted in a room mirror, or, mounted in the instrument panel 20. An image of a face of the driver obtained from the image sensor 80 may be used to identify an identity of the driver, detect a motion (specifically, eye blinking) of the eye of the driver, or detect an angle of the face of the driver.

A brake pedal and a gas pedal 90 are disposed below the steering wheel 10 and sensors may be attached to the brake pedal and the gas pedal 90. When the driver steps on the brake pedal or the gas pedal 90, the brake pedal sensor or the gas pedal sensor detects a displacement and/or an angle of the brake pedal or the gas pedal 90.

The driver's seat 100 may include a headrest 110 and a massage pad 120. The headrest 110 may include a headrest sensor 115 located in a place where a head of the driver is rested. The headrest sensor 110 may include a pressure sensor which detects a pressure applied to the headrest 110 by the head of the driver or a proximity sensor which detects a distance between the head of the driver and the headrest 110.

The massage pad 120 may include a plurality of actuators 125 which is embedded in the driver's seat 100 to apply a pressure (acupressure) and/or vibration to the back of the driver. The massage pad 120 may operate the plurality of actuators 125 in accordance with a control input from the user or a control signal from the control device.

A window 130 through which outside air flows in the vehicle is mounted in the door of the driver's seat and the window 130 may be open/closed in accordance with the manual control of the user or a control signal of the control device.

Even though not illustrated in FIG. 1 for the purpose of simplification, various devices required for the driving of the vehicle or the convenience of the driver may be further disposed in the internal environment of the vehicle.

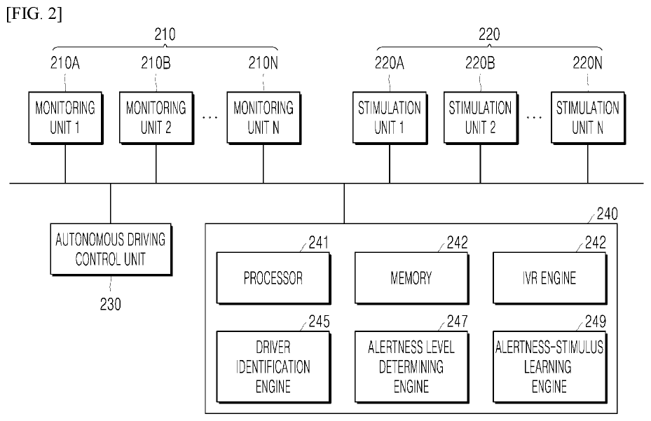

FIG. 2 is a block diagram schematically illustrating an in-car system for preventing the drowsy driving according to an exemplary embodiment of the present disclosure.

Referring to FIG. 2, the in-car system may include a plurality of monitoring units 210, a plurality of stimulation units 220, an autonomous driving control unit 230, and a control device 240.

According to an exemplary embodiment, the monitoring units 210, the stimulation units 220, the autonomous driving control unit 230, and the control device 240 may be electrically connected to communicate with each other, for example, through an in-car communication bus 250. According to another exemplary embodiment, the monitoring units 210, the stimulation units 220, the autonomous driving control unit 230, and the control device 240 may be communicably connected with each other through a wireless communication technology. The wireless communication technology may include one or more of a fifth generation (5G) cellular network, Bluetooth, Infrared data association (IrDA), Internet of Things (IoT), local area network (LAN), low power network (LPN), low power wide area network (LPWAN), personal area network (PAN), radio frequency identification (RFID), ultra-wide band (UWB), wireless fidelity (Wi-Fi), wireless LAN (WLAN), or ZigBee communication technologies, but is not limited thereto.

Even though not illustrated in FIG. 2 for the purpose of simplification, the in-car system may further include various devices required for the operation of the vehicle, for example, an engine control unit, a transmission control unit, a brake control unit, and a battery control unit.

The monitoring unit 210 includes any device for monitoring a state of the driver. For example, the monitoring unit 210 may include at least one of the biometric sensor 13 of the steering wheel 10, the image sensor 80, the brake pedal sensor, the gas pedal sensor, or the headrest sensor 115 illustrated in FIG. 1. However, the monitoring unit 210 is not limited to the above-described examples, but any device which is capable of monitoring the state of the driver may be used as the monitoring unit 210.

The monitoring unit 210 transmits acquired or detected information to the control device 240. The information from the monitoring unit 210 may be used to determine an alertness or a drowsy level of the driver. For example, one or a combination of the heart rate and/or oxygen saturation of the driver detected by the biometric sensor 13, a movement of the eye of the driver or an angle of the face of the driver acquired by the image sensor 80, an abnormal movement of a pedal detected by the brake pedal sensor or the gas pedal sensor, or nodding of the driver's head detected by the headrest sensor 115 may be used as a factor for determining the alertness or the drowsy level of the driver.

The stimulation unit 220 may include any device which delivers any type of stimulus (for example, a visual stimulus, an auditory stimulus, or a tactile stimulus) to the driver. For example, the stimulation unit 220 may include at least one of the vibration unit 15 of the steering wheel 10, the air conditioner 30, the display screen 40, the speaker 50, the interior light 60, the massage pad 120 of the driver's seat 100, or the window 130 illustrated in FIG. 1. However, the stimulation unit 220 is not limited to the above-described examples, but any device capable of delivering the stimulus to the driver may be used as the stimulation unit 220.

In order to improve the alertness of the driver, the stimulation unit 220 may operate to deliver the stimulus to the driver in accordance with the control signal from the control device 240. For example, the vibration unit 15 applies a vibration to a hand of the driver in accordance with the control signal from the control device 240 to increase the alertness of the driver. The massage pad 120 of the seat 100 may apply a vibration or a pressure to the back of the driver in accordance with the control signal from the control device 240 to increase the alertness of the driver. The air conditioner 30 blows cooled air or heated air in accordance with the control signal from the control device 240 to energize the driver. The window 130 is open to allow the inflow of outside air in accordance with the control signal from the control device 240 to energize the driver. The speaker 50 outputs music, a voice, or a warning sound in accordance with the control signal from the control device 240 to deliver the auditory stimulus to the driver. The display screen 40 or the interior light 60 outputs a flickering screen or red light which draws the driver's attention in accordance with the control signal from the control device 240 to deliver the visual stimulus to the driver.

Although the monitoring unit 210 and the stimulation unit 220 are illustrated with reference to FIG. 1, the monitoring unit 210 or the stimulation unit 220 is not limited to the above-described examples, and any monitoring unit or stimulation unit known to those skilled in the automotive manufacturing field may be used.

The autonomous driving control unit 230 is configured to control a plurality of vehicle control units (for example, an engine control unit, a transmission control unit, a brake control unit, or a steering control unit) using a plurality of sensors for autonomous driving (for example, a laser scanner, an ultrasonic sensor, a 360-degree camera, a front camera, a short range radar, a medium range radar, or a long range radar). The autonomous driving control unit 230 may have any configuration known to those skilled in the automotive manufacturing field.

The autonomous driving control unit 230 may control the vehicle at one of SAE levels defined by Society of Automotive Engineering (SAE) upon a request from the driver or the control device 240. The SAE levels include a no automation level (level 0), a driver assistance level (level 1), a partial automation level (level 2), a conditional automation level (level 3), a high automation level (level 4), and a full automation level (level 5).

That is, the autonomous driving control unit 230 may assist the speed of the vehicle or the steering control by the driver (adaptive cruise or assistance to keep the lane), directly control the speed of the vehicle or the steering without being supported by the driver (autopilot or automatic parking), or control the braking or steering of the vehicle regardless of the control of the driver (automatic emergency braking or steering).

The control device 240 may include a processor 241, a memory 242, an interactive voice response (IVR) engine 243, a driver identification engine 245, an alertness level determining engine 247, and an alertness-stimulus learning engine 249. The control device 240 may be combined in an electronic control unit in the vehicle or provided separately from the electronic control unit.

The processor 241 may be any data processing device which is implemented as hardware and has a circuit structured to perform functions expressed by codes or commands included in a program stored in the memory 242. For example, the processor 241 may include one or more of a microprocessor, a central processing unit (CPU), a processor core, a multi-processor, an application-specific integrated circuit (ASIC), and a field programmable gate array (FPGA), but is not limited thereto. The processor 241 performs operations of the control device 240 in accordance with a program stored in the memory 242. Hereinafter, it is understood that unless explicitly described, operations of the control device 240 may be performed by the processor 241.

The memory 242 may be a tangible computer-readable medium which stores a computer program to be executed by the processor 241. The memory 242 further stores information on identities of a plurality of drivers. For example, the memory 242 may include one or more of a magnetic medium such as a hard disk, a floppy disk, or a magnetic tape, an optical recording medium such as a CD-ROM or a DVD, a magneto-optical medium such as a floptical disk, and a solid-state semiconductor device such as a RAM, a ROM, or a flash memory, but is not limited thereto. Further, the memory 242 may include one or more of a volatile memory and a non-volatile memory.

The IVR engine 243 may include an artificial neural network (ANN) which has been trained to analyze a meaning of a voice from the acoustic sensor 70 and output a signal or a natural language related to the analyzed meaning, using a deep learning technology. According to another exemplary embodiment, the IVR engine 243 may communicate with the artificial neural network which is remotely located. The IVR engine 243 analyzes a voice command of the driver to cause the control device 240 to perform an operation in response to the voice command or outputs an answer to a voice question of the user, or asks a question to the driver to induce an answer of the driver.

The driver identification engine 245 may include an artificial neural network which has been trained to analyze image information from the image sensor 80 and identify the identity of the driver, using a deep learning technology. For example, the driver identification engine 245 may have been trained to calculate a possibility that a person appearing in an image from the image sensor 80 is an existing driver A or B stored in the memory 242, or a third party which is not stored in the memory 242.

The alertness level determining engine 247 may include or communicate with an artificial neural network which has been trained to analyze information from one or more monitoring units 210 and determine an alertness level of the driver, using a deep learning technology. For example, the alertness level determining engine 247 may have been trained to determine the alertness level of the driver with one or more of the heart rate and/or oxygen saturation information of the driver detected by the biometric sensor 13, the image (specifically, an image of the face and an image of the eye) of the driver acquired by the image sensor 80, a displacement or an angle of the brake pedal or the gas pedal 90, and distance information between the head of the driver and the headrest 110 detected by the headrest sensor 115 as input data.

The alertness-stimulus learning engine 249 may include an artificial neural network which has been trained to identify a stimulus suitable to improve the alertness of the driver depending on the identity and the alertness level of the driver, among a plurality of available stimuli, for example, using reinforcement learning. According to an exemplary embodiment, the alertness-stimulus learning engine 249 outputs one of available stimuli, depending on the identity and the alertness level of the driver. According to another exemplary embodiment, the alertness-stimulus learning engine 249 may output a list of a plurality of stimuli to which scores are assigned.

The IVR engine 243, the driver identification engine 245, the alertness level determining engine 247, and the alertness-stimulus learning engine 249 may include an artificial neural network (ANN) configured to generate an output for each provided input using a deep learning technique and may be implemented as a hardware module and/or software module.

An ANN is a data processing system modelled after the mechanism of biological neurons and interneuron connections, in which a number of neurons, referred to as nodes or processing elements, are interconnected in layers. ANNs are models used in machine learning and may include statistical learning algorithms conceived from biological neural networks (particularly of the brain in the central nervous system of an animal) in machine learning and cognitive science. ANNs may refer generally to models that have artificial neurons (nodes) forming a network through synaptic interconnections, and acquires problem-solving capability as the strengths of synaptic interconnections are adjusted throughout training. An ANN may include a number of layers, each including a number of neurons. Furthermore, the ANN may include synapses that connect the neurons to one another.

An ANN may be defined by the following three factors: (1) a connection pattern between neurons on different layers; (2) a learning process that updates synaptic weights; and (3) an activation function generating an output value from a weighted sum of inputs received from a previous layer.

An ANN may include a deep neural network (DNN). Specific examples of the DNN include a convolutional neural network (CNN), a recurrent neural network (RNN), a deep belief network (DBN), and the like, but are not limited thereto.

An ANN may be classified as a single-layer neural network or a multi-layer neural network, based on the number of layers therein. In general, a single-layer neural network may include an input layer and an output layer. In general, a multi-layer neural network may include an input layer, one or more hidden layers, and an output layer.

The input layer receives data from an external source, and the number of neurons in the input layer is identical to the number of input variables. The hidden layer is located between the input layer and the output layer, and receives signals from the input layer, extracts features, and feeds the extracted features to the output layer. The output layer receives a signal from the hidden layer and outputs an output value based on the received signal. Input signals between the neurons are summed together after being multiplied by corresponding connection strengths (synaptic weights), and if this sum exceeds a threshold value of a corresponding neuron, the neuron can be activated and output an output value obtained through an activation function.

A deep neural network with a plurality of hidden layers between the input layer and the output layer may be the most representative type of artificial neural network which enables deep learning, which is one machine learning technique.

An ANN can be trained using training data. Here, the training may refer to the process of determining parameters of the artificial neural network by using the training data, to perform tasks such as classification, regression analysis, and clustering of input data. Such parameters of the artificial neural network may include synaptic weights and biases applied to neurons.

An artificial neural network trained using training data can classify or cluster input data according to a pattern within the inputt data.

Throughout the present specification, an artificial neural network trained using training data may be referred to as a trained model.

Hereinbelow, learning paradigms of an artificial neural network will be described in detail.

Learning paradigms, in which an artificial neural network operates, may be classified into supervised learning, unsupervised learning, semi-supervised learning, and reinforcement learning.

Supervised learning is a machine learning method that derives a single function from the training data.

Among the functions that may be thus derived, a function that outputs a continuous range of values may be referred to as a regressor, and a function that predicts and outputs the class of an input vector may be referred to as a classifier.

In supervised learning, an artificial neural network can be trained with training data that has been given a label.

Here, the label may refer to a target answer (or a result value) to be guessed by the artificial neural network when the training data is input to the artificial neural network.

Throughout the present specification, the target answer (or a result value) to be guessed by the artificial neural network when the training data is input may be referred to as a label or labeling data.

Throughout the present specification, assigning one or more labels to training data in order to train an artificial neural network may be referred to as labeling the training data with labeling data.

Training data and labels corresponding to the training data together may form a single training set, and as such, they may be input to an artificial neural network as a training set.

The training data may exhibit a number of features, and the training data being labeled with the labels may be interpreted as the features exhibited by the training data being labeled with the labels. In this case, the training data may represent a feature of an input object as a vector.

Using training data and labeling data together, the artificial neural network may derive a correlation function between the training data and the labeling data. Then, through evaluation of the function derived from the artificial neural network, a parameter of the artificial neural network may be determined (optimized).

Unsupervised learning is a machine learning method that learns from training data that has not been given a label.

More specifically, unsupervised learning may be a training scheme that trains an artificial neural network to discover a pattern within given training data and perform classification by using the discovered pattern, rather than by using a correlation between given training data and labels corresponding to the given training data.

Examples of unsupervised learning include, but are not limited to, clustering and independent component analysis.

Examples of artificial neural networks using unsupervised learning include, but are not limited to, a generative adversarial network (GAN) and an autoencoder (AE).

GAN is a machine learning method in which two different artificial intelligences, a generator and a discriminator, improve performance through competing with each other.

The generator may be a model generating new data that generates new data based on true data.

The discriminator may be a model recognizing patterns in data that determines whether input data is from the true data or from the new data generated by the generator.

Furthermore, the generator may receive and learn from data that has failed to fool the discriminator, while the discriminator may receive and learn from data that has succeeded in fooling the generator. Accordingly, the generator may evolve so as to fool the discriminator as effectively as possible, while the discriminator evolves so as to distinguish, as effectively as possible, between the true data and the data generated by the generator.

An auto-encoder (AE) is a neural network which aims to reconstruct its input as output.

More specifically, AE may include an input layer, at least one hidden layer, and an output layer.

Since the number of nodes in the hidden layer is smaller than the number of nodes in the input layer, the dimensionality of data is reduced, thus leading to data compression or encoding.

Furthermore, the data output from the hidden layer may be input to the output layer. Given that the number of nodes in the output layer is greater than the number of nodes in the hidden layer, the dimensionality of the data increases, thus leading to data decompression or decoding.

Furthermore, in the AE, the input data is represented as hidden layer data as interneuron connection strengths are adjusted through training. The fact that when representing information, the hidden layer is able to reconstruct the input data as output by using fewer neurons than the input layer may indicate that the hidden layer has discovered a hidden pattern in the input data and is using the discovered hidden pattern to represent the information.

Semi-supervised learning is a machine learning method that makes use of both labeled training data and unlabeled training data.

One semi-supervised learning technique involves reasoning the label of unlabeled training data, and then using this reasoned label for learning. This technique may be used advantageously when the cost associated with the labeling process is high.

Reinforcement learning may be based on a theory that given the condition under which a reinforcement learning agent can determine what action to choose at each time instance, the agent can find an optimal path to a solution solely based on experience without reference to data.

Reinforcement learning may be performed mainly through a Markov decision process.

Markov decision process consists of four stages: first, an agent is given a condition containing information required for performing a next action; second, how the agent behaves in the condition is defined; third, which actions the agent should choose to get rewards and which actions to choose to get penalties are defined; and fourth, the agent iterates until future reward is maximized, thereby deriving an optimal policy.

An artificial neural network is characterized by features of its model, the features including an activation function, a loss function or cost function, a learning algorithm, an optimization algorithm, and so forth. Also, the hyperparameters are set before learning, and model parameters can be set through learning to specify the architecture of the artificial neural network.

For instance, the structure of an artificial neural network may be determined by a number of factors, including the number of hidden layers, the number of hidden nodes included in each hidden layer, input feature vectors, target feature vectors, and so forth.

Hyperparameters may include various parameters which need to be initially set for learning, much like the initial values of model parameters. Also, the model parameters may include various parameters sought to be determined through learning.

For instance, the hyperparameters may include initial values of weights and biases between nodes, mini-batch size, iteration number, learning rate, and so forth. Furthermore, the model parameters may include a weight between nodes, a bias between nodes, and so forth.

Loss function may be used as an index (reference) in determining an optimal model parameter during the learning process of an artificial neural network. Learning in the artificial neural network involves a process of adjusting model parameters so as to reduce the loss function, and the purpose of learning may be to determine the model parameters that minimize the loss function.

Loss functions typically use means squared error (MSE) or cross entropy error (CEE), but the present disclosure is not limited thereto.

Cross-entropy error may be used when a true label is one-hot encoded. One-hot encoding may include an encoding method in which among given neurons, only those corresponding to a target answer are given 1 as a true label value, while those neurons that do not correspond to the target answer are given 0 as a true label value.

In machine learning or deep learning, learning optimization algorithms may be deployed to minimize a loss function, and examples of such learning optimization algorithms include gradient descent (GD), stochastic gradient descent (SGD), momentum, Nesterov accelerate gradient (NAG), Adagrad, AdaDelta, RMSProp, Adam, and Nadam.

GD includes a method that adjusts model parameters in a direction that decreases the value of a loss function by using a current slope of the loss function.

The direction in which the model parameters are to be adjusted may be referred to as a step direction, and a size by which the model parameters are to be adjusted may be referred to as a step size.

Here, the step size may mean a learning rate.

GD obtains a slope of the loss function through use of partial differential equations, using each of model parameters, and updates the model parameters by adjusting the model parameters by a learning rate in the direction of the obtained slope.

SGD may include a method that separates the training dataset into mini-batches, and by performing gradient descent for each of these mini batches, increases the frequency of gradient descent.

Adagrad, AdaDelta and RMSProp may include methods that increase optimization accuracy in SGD by adjusting the step size, and may also include methods that increase optimization accuracy in SGD by adjusting the momentum and step direction. Adam may include a method that combines momentum and RMSProp and increases optimization accuracy in SGD by adjusting the step size and step direction. Nadam may include a method that combines NAG and RMSProp and increases optimization accuracy by adjusting the step size and step direction.

Learning rate and accuracy of an artificial neural network rely not only on the structure and learning optimization algorithms of the artificial neural network but also on the hyperparameters thereof. Therefore, in order to obtain a good learning model, it is important to choose a proper structure and learning algorithms for the artificial neural network, but also to choose proper hyperparameters.

In general, the artificial neural network is first trained by experimentally setting hyperparameters to various values, and based on the results of training, the hyperparameters can be set to optimal values that provide a stable learning rate and accuracy.

Meanwhile, the artificial neural network can be trained by adjusting connection weights between nodes (if necessary, adjusting bias values as well) so as to produce desired output from given input. Also, the artificial neural network can continuously update the weight values through learning. Furthermore, methods such as back propagation may be used in training the artificial neural network.

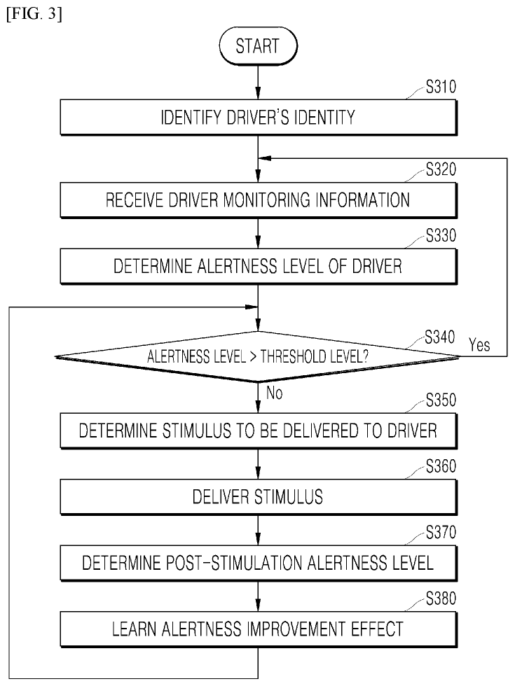

FIG. 3 is a flowchart illustrating an exemplary method of improving an alertness of a driver according to an exemplary embodiment of the present disclosure. The method of improving an alertness of a driver according to an exemplary embodiment of the present disclosure will be described below with reference to FIG. 3.

### Identifying Identity of Driver

In step S310, the control device 240 identifies the identity of the driver. For example, the driver may select one of a plurality of driver's identities stored in the memory 242 using the control button 11 and the control device 240 identifies the identity of the driver by means of the selection of the driver.

In another example, when the vehicle is started, the control device 240 may identify the identity of the driver from an image of the driver, particularly of the face, acquired by the image sensor 80. The control device 240 provides the image of the driver received from the image sensor 80 to the driver identification engine 245 as input data. The driver identification engine 245 analyzes the image of the driver's face to output result data which numerically represent possibilities that the driver matches the respective plurality of identities stored in the memory 242 or the driver corresponds to a new identity which is not stored in the memory 242. The control device 240 may determine an identity having the highest possibility from the result data output from the driver identification engine 245 as an identity of the driver.

### Receiving Driver Monitoring Information

In step S320, the control device 240 receives driver monitoring information from one or more monitoring units 210. The driver monitoring information may include at least one of a heart rate of the driver, oxygen saturation, a face image of the driver, a displacement or angle of the brake pedal or the gas pedal, a distance between the head of the driver and the headrest, or a pressure applied to the headrest by the head of the driver, but is not limited thereto.

The monitoring units 210A, 210B, and 210N may consistently monitor the state of the driver during driving of the vehicle. Each monitoring unit 210 may inform the control device 240 of the monitored driver's state at a predetermined interval of time or upon the request of the control device 240.

The biometric sensor 13 may inform the control device 240 of the heart rate and the oxygen saturation of the driver, for example, at an interval of time of 10 seconds. The image sensor 80 may stream the acquired driver's image to the control device 240 in a real time. The brake pedal sensor and the gas pedal sensor may inform the control device 240 of the displacement or the angle of the brake pedal and the gas pedal 90, for example, at an interval of time of 0.1 second. The headrest sensor 115 may inform the control device 240 of the distance between the head of the driver and the headrest 110 or a pressure applied to the headrest by the head of the driver, for example, at an interval of time of one second.

### Determining Alertness Level of Driver

In step S330, the control device 240 determines an alertness level of the driver based on the driver monitoring information. For example, the control device 240 provides one or more items of the driving monitoring information received from the monitoring unit 210 to the alertness level determining engine 247 as input data. The alertness level determining engine 247 analyzes the input data and numerically represents the alertness of the driver to output an alertness level.

For example, the alertness level determining engine 247 detects how much or how often the eyes are closed, from the face image of the driver and numerically represents the alertness of the driver therefrom. In another example, the alertness level determining engine 247 detects an angle of the driver's face o with respect to the front direction and numerically represents the alertness of the driver therefrom. Further, in another example, the alertness level determining engine 247 may numerically represent the alertness of the driver from a profile of a distance between the head of the driver and the headrest 110 over time. Further, in another example, the alertness level determining engine 247 may numerically represent the alertness of the driver from a profile of a displacement of the brake pedal and the gas pedal 90 over time.

The alertness level determining engine 247 weights result values numerically represented as a result of the above-described factors with different weights and determines the alertness level of the driver from a computation (for example, sum) of the weighted result value. For example, an alertness value based on the image sensor 80 may be weighted with the highest weight and an alertness value based on the displacement profile of the brake pedal or the gas pedal 90 may be weighted with the lowest weight.

The alertness level of the driver may be determined as a value between 0 and 100% or may be determined as a value in any range. Hereinafter, for the sake of convenience, the alertness level of the driver is exemplified to have a value between 0 and 5. For example, an alertness level between 0 and 1 may be “consistently unconsciousness”, an alertness level between 1 and 2 may be “intermittently unconsciousness”, an alertness level between 2 and 3 may be “imminent drowsiness”, an alertness level between 3 and 4 may be “reduced concentration”, and an alertness level between 4 and 5 may be a “sufficient alertness” state

### Comparing Alertness Level of Driver and Threshold Level

In step S340, the control device 240 compares the alertness level of the driver with a threshold level. The threshold level is a predetermined value to determine a possibility of drowsy driving by the driver. For example, when the alertness level is determined to be a value between 1 and 5, the threshold level may be predetermined to be 3.5.

When the alertness level of the driver exceeds the threshold level, there is no need to take an action to improve the alertness of the driver. Therefore, the process returns to step S320 of receiving the driver monitoring information and steps S320 to 340 are repeated.

When the alertness level of the driver is equal to or lower than the threshold level (that is, it is determined that there is a possibility of drowsy driving), it is necessary to take the following action to improve the alertness of the driver.

### Determining Stimulus to be Delivered to Driver

In step S350, the control device 240 determines a stimulus to be delivered to the driver based on the identity of the driver and the alertness level of the driver. For example, the control device 240 provides the identity of the driver and the alertness level of the driver to the alertness-stimulus learning engine 249 as input data and determines a stimulus to be delivered to the driver based on output data from the alertness-stimulus learning engine 249.

Table 1 shows types of exemplary stimuli, delivering units, and properties which may be selected in the vehicle according to an exemplary embodiment of the present disclosure.

TABLE 1

| Stimulus delivering unit | Stimulus type | Property of stimulus |
|---|---|---|
| Vibration unit 15 of | Vibration | Intensity, Pattern |
| steering wheel 10 | | |
| Air conditioner 30 | Air blowing | Temperature, Fan speed |
| Display screen 40 | Warning screen | Color, Brightness, |
| | | Flickering |
| Speaker 50 | Music/Warning | Genre, Volume |
| | sound | |
| IVR engine 243/Speaker | Interactive | Subject of conversation |
| 50 | conversation | |
| Interior light 60 | Light | Color, Brightness, |
| | | Flickering |
| Massage pad 120 | Vibration/ | Intensity, Pattern |
| | Acupressure | |
| Window 130 | Air inflow | Window opening degree |

A stimulus effective to improve the alertness may vary depending on the drivers and/or the alertness levels. When the same stimulus is applied to different drivers, the alertness effect of the stimulus may vary depending on the drivers. Further, even though the same stimulus is applied to same driver, an alertness effect of the stimulus may vary depending on an alertness level of the driver.

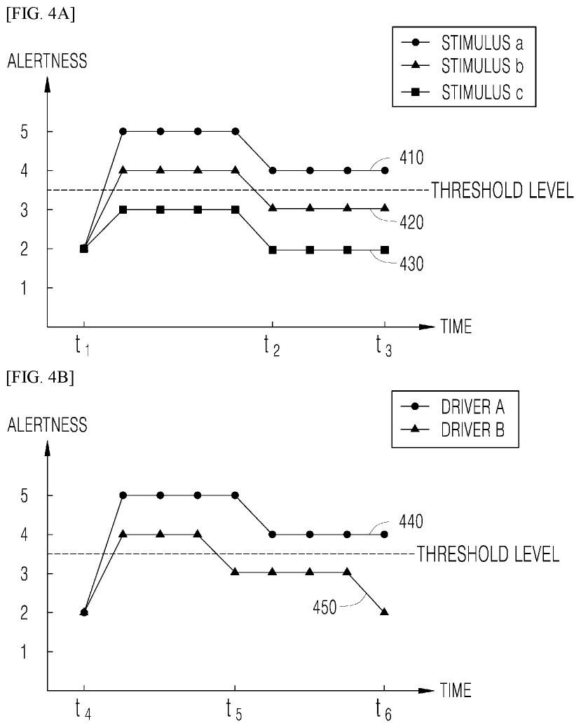

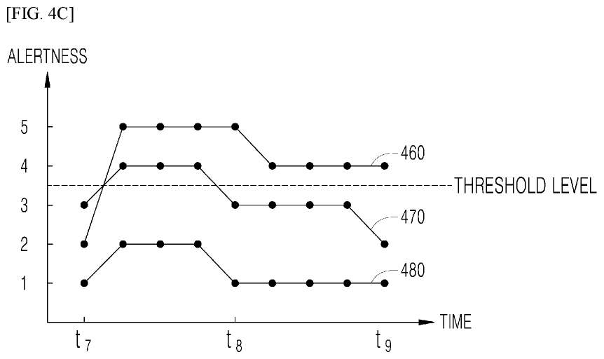

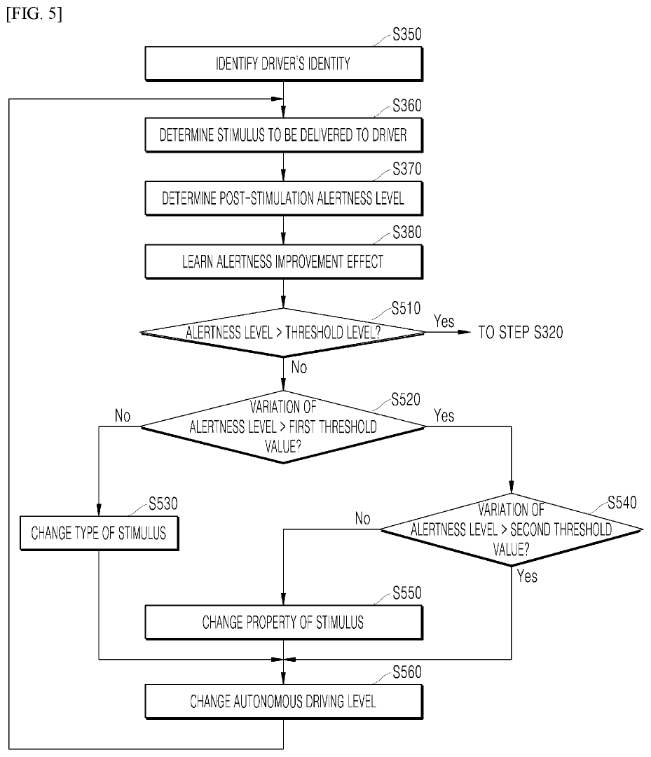

FIGS. 4A to 4C illustrate exemplary graphs showing a change in alertness of a driver over time after applying a stimulus.

FIG. 4A is an exemplary graph illustrating a change in alertness of a driver over time after applying a stimulus according to a type of stimulus. At first, the driver A is in an alertness level 2. The solid line 410 represents an example in which a stimulus a (for example, a music play through the speaker 50) is applied to a driver at a timepoint t1. According to the solid line 410, the alertness level of the driver is increased to exceed the threshold level immediately after the timepoint t1 and is maintained to be higher than the threshold level until the timepoint t3. The solid line 420 represents an example in which a stimulus b (for example, vibration by the vibration unit 15) is applied to the same driver at the timepoint t1. According to the solid line 420, the alertness level of the driver is increased to exceed the threshold level immediately after the timepoint t1, but drops to be equal to or lower than the threshold level at a timepoint t2. The solid line 430 represents an example in which a stimulus c (for example, flickering light is emitted through the interior light 60) is applied to the driver at a timepoint t1. According to the solid line 430, the alertness level of the driver is slightly increased immediately after the timepoint t1, but does not exceed the threshold level. It is understood from FIG. 4A that different types of stimuli show different alertness effects.

FIG. 4B is a graph illustrating a change in an alertness of a driver over time after applying stimulus when the same stimulus is applied to different drivers. At first, both the driver A and the driver B are in an alertness level 2. The solid line 440 represents an example in which a stimulus a is applied to the driver A at a timepoint t4. According to the solid line 440, the alertness level of the driver A is increased to exceed the threshold level immediately after the timepoint t4 and is maintained to be higher than the threshold level until the timepoint t6. The solid line 450 represents an example in which the same stimulus a is applied to the driver B at a timepoint t4. According to the solid line 450, the alertness level of the driver B is increased to exceed the threshold level immediately after the timepoint t4, but drops to be lower than the threshold level at a timepoint t5. It is understood from FIG. 4B that even the same stimulus may show different alertness effects for different drivers.

FIG. 4C is an exemplary graph illustrating a change in an alertness of a driver over time after applying stimulus when the same stimulus is applied to the same driver. The solid line 460 represents an example in which a stimulus a is applied to the driver A which is in an alertness level 2 at a timepoint t7. According to the solid line 460, the alertness level of the driver A is increased to exceed the threshold level immediately after the timepoint t7 and is maintained to be higher than the threshold level until the timepoint t9. The solid line 470 represents an example in which a stimulus a is applied to the driver A which is in an alertness level 3 at a timepoint t7. According to the solid line 470, the alertness level of the driver A is increased to exceed the threshold level immediately after the timepoint t7, but drops to be lower than the threshold level at a timepoint t8. The solid line 480 represents an example in which a stimulus a is applied to the driver A which is in an alertness level 1 at a timepoint t7. According to the solid line 480, the alertness level of the driver A is slightly increased immediately after the timepoint t7, but does not exceed the threshold level. It is understood from FIG. 4C that even though the same stimulus is applied to the same driver, the alertness effect may vary depending on the alertness level of the driver when the stimulus is applied.

The alertness-stimulus learning engine 249 may determine an effective stimulus depending on the identity and the alertness level of the driver. According to an exemplary embodiment, when the identify and the alertness level of the driver are given as status data, the alertness-stimulus learning engine 249 may output a list of available stimuli together with the scores of the stimuli.

For example, when the driver A is in an alertness level 2, the alertness-stimulus learning engine 249 may assign the highest score to the music play and assign a second highest score to the vibration by the vibration unit 15 of the steering wheel 10. When the same driver A is in an alertness level 3, for example, the alertness-stimulus learning engine 249 may assign the highest score to the interactive conversation using the IVR engine 243 and assign the second highest score to application of vibration and/or acupressure using the massage pad 120.

The control device 240 may determine a stimulus to be delivered to the driver based on the scores of the stimuli output from the alertness-stimulus learning engine 249. For example, when the driver A is in an alertness level 2, the control device 240 may determine the music play assigned with the highest score as a stimulus to be delivered to the driver.

### Delivering Stimulus to Driver and Autonomous Driving Control

In step S360, the control device 240 instructs one or more stimulation units 220 to deliver the determined stimulus. For example, the control device 240 may output a control signal which operates the massage pad 120 of the seat 100. The stimulation unit 220 which receives the control signal operates to provide a stimulus to the driver. According to an exemplary embodiment, since the stimulus to be delivered to the driver is determined based on both the identity of the driver and the alertness level of the driver, the alertness of the driver can be improved in a driver-specific manner.

When the stimulus is applied to the driver, the driver may be surprised at the stimulus and there is a possibility that the driver may unintentionally make a sudden steering change. In order to prevent the unintentional steering change, the control device 240 may request the autonomous driving control unit 230 to perform at least a lane keeping function prior to applying the stimulus to the driver.

In addition, the control device 240 may transmit a request for the autonomous driving to the autonomous driving control unit 230 together with applying the stimulus to the driver. The requested autonomous driving level may vary depending on the alertness levels of the driver. For example, when the alertness level of the driver is within a range of 0 to 1, the control device 240 requests the autonomous control unit 240 to perform the autonomous driving control at an SAE level 4 or higher. When the alertness level of the driver is within a range of 1 to 2, the control device 240 requests the autonomous driving control at an SAE level 3. When the alertness level of the driver is within a range of 2 to 3, the control device 240 requests the autonomous driving control at an SAE level 2.

### Determining Post-Stimulation Alertness Level

In step S370, a post-stimulation alertness level of the driver is determined. Similarly to step S320, the control device 240 receives the driver monitoring information from one or more monitoring units 210 and similarly to step S330, the control device 240 determines the alertness level of the driver based on the driver monitoring information.

It may take some time for the alertness of the driver to be changed after delivering the stimulus to the driver. Therefore, the control device 240 may determine the post-stimulation alertness level of the driver after a predetermined time has elapsed since the stimulus is delivered to the driver. In this case, the predetermined time may vary depending on the stimuli delivered to the driver.

For example, since the driver may immediately respond to the warning sound the control device 240 may determine the post-stimulation alertness level 5 minutes after outputting the warning sound. When the vibration or acupressure is applied through the massage pad 120, the control device 240 may determine the post-stimulation alertness level 30 seconds after delivering the stimulus. In the case of the interactive conversation through the IVR engine 243, the control device 240 may determine the post-stimulation alertness level one minute after starting the conversation.

### Learning Alertness Improvement Performance on Stimulus

In step S380, the alertness-stimulus learning engine 249 learns the performance of the stimulus. According to an exemplary embodiment, the control device 240 may feedback a post-stimulation alertness level of the driver to the alertness-stimulus learning engine 249 together with the information on the stimulus delivered to the driver, as training data. When the post-simulation alertness level is increased as compared with the alertness level before delivering the stimulus, the control device 240 may provide a reward to the alertness-stimulus learning engine 249 together with the training data. In contrast, when the post-stimulation alertness level is reduced as compared with that before delivering the stimulus, the control device 240 may provide a penalty to the alertness-stimulus learning engine 249 together with the training data.

According to another exemplary embodiment, the control device 240 may feedback a variation of alertness level before and after delivering the stimulus to the alertness-stimulus learning engine 249 together with the information on the stimulus delivered to the driver, as training data. First, the control device 240 determines the variation of the alertness level of the driver by the stimulus delivery in step S360. The variation of the alertness level may be a difference between the post-stimulation alertness level determined in step S370 and the alertness level before delivering the stimulus in step S360. The variation of the alertness level refers to a performance of the stimulus delivered to the driver in step S360. The control device 240 provides a reward or a penalty determined based on the variation of the alertness level to the alertness-stimulus learning engine 249 together with the variation of the alertness level. When the alertness level of the driver is significantly improved, the alertness-stimulus learning engine 249 may receive a large reward for the stimulus. When the alertness level of the driver is slightly improved, the alertness-stimulus learning engine 249 may receive a small reward for the stimulus.

According to another exemplary embodiment, the control device 240 may provide the reward for the stimulus to the alertness-stimulus learning engine 249 based on a time when the improvement of the alertness of the driver is maintained. For example, when the improvement of the alertness for the stimulus is maintained for a long time, the alertness-stimulus learning engine 249 may receive a large reward for the stimulus. When the improvement of the alertness for the stimulus is maintained for a short time, the alertness-stimulus learning engine 249 may receive a small reward for the stimulus.

The alertness-stimulus learning engine 249 may learn the performance of the stimulus that was output in accordance with the identity and the alertness level of the driver by the reward or the penalty. When the identity and the alertness level of the same driver are given later, the alertness-stimulus learning engine 249 may output the list of stimuli with an increased score for the stimulus which was given the reward or a decreased score for the stimulus which was given the penalty to output the list of stimuli.

Thereafter, the process returns step S340 to compare the post-stimulation alertness level determined in step S370 with the threshold level again and the steps S320 to 380 may be repeated until the driving ends.

FIG. 5 is a flowchart illustrating an exemplary method of improving an alertness of a driver according to another exemplary embodiment of the present disclosure. According to this exemplary embodiment, only the process after step S380 of learning the improvement effect of the alertness for the stimulus is different from that of the exemplary embodiment illustrated in FIG. 3 and the steps S310 to S380 are the same as those in the exemplary embodiment illustrated in FIG. 3. In FIG. 5, steps S310 to S340 which are the same steps as those in FIG. 3 will be omitted. Hereinafter, the descriptions of the same steps S310 to S380 will be omitted.

### Comparing Alertness Level of Driver and Threshold Level

In step S510, the control device 240 compares the post-stimulation alertness level of the driver determined in step S370 with a predetermined threshold level. When the post-stimulation alertness level of the driver exceeds the threshold level, it is understood that the driver is out of the drowsy driving. Therefore, when the post-stimulation alertness level of the driver exceeds the threshold level, there is no need to take an action to improve the alertness and the process returns to step S320 of receiving driver monitoring information.

When the post-stimulation alertness level of the driver is still equal to or lower than the threshold level (that is, it is determined that there is a possibility of drowsy driving), it is necessary to take the following action to improve the alertness of the driver.

### Comparing Variation of Alertness Level and First Threshold Value

In step S520, the control device 240 compares a variation of the post-stimulation alertness level of the driver determined in step S380 with the first threshold level. The first threshold value is a value which is predetermined to determine whether the change of the post-stimulation alertness level is a significant change. The control device 240 determines whether the variation of the post-stimulation alertness level exceeds the first threshold value to determine whether the stimulus delivered to the driver has a positive effect to improve the alertness of the driver.

### Changing Type of Stimulus

When the variation of the post-stimulation alertness level does not exceed the first threshold value, it is understood that the type of stimulus previously delivered to the driver does not show a sufficient performance to improve the alertness of the driver. Accordingly, when the variation of the post-stimulation alertness level does not exceed the first threshold value, the control device 240 may determine a stimulus which is of a different type from that of the previously delivered stimulus as a stimulus to be delivered to the driver in step S530.

For example, the control device 240 may determine a stimulus having a second highest score from data output by the alertness-stimulus learning engine 249 in step S350 as a type of stimulus which will be newly delivered to the driver. In another example, the control device 240 inputs new state data to the alertness-stimulus learning engine 249 and determines a type of a stimulus to be newly delivered based on output data from the alertness-stimulus learning engine 249.

### Comparing Variation of Alertness Level and Second Threshold Value

When the variation of the post-stimulation alertness level exceeds the first threshold value, in step S540, the control device 240 compares the variation of alertness level with a second threshold value which is predetermined. When the variation of alertness level exceeds the second threshold value, it is understood that the stimulus which is previously delivered to the driver shows a sufficient performance to improve the alertness of the driver. Therefore, when the variation of post-stimulation alertness level exceeds the second threshold value, the type and the detail attribution of stimulus are maintained as it is without being changed.

Changing Property while Maintaining Type of Stimulus

When the variation of post-stimulation alertness level exceeds the first threshold value, but does not exceed the second threshold value, the control device 240 may change the property of the stimulus while maintaining the same type of stimulus as a stimulus to be delivered to the driver in step S550.

Changing the property of the stimulus may be, for example, changing a vibration intensity or a vibration pattern of the vibration unit 15, changing a volume of the speaker 50, or changing a subject of the conversation for the interactive conversation. For example, when an auditory stimulus (music play) is previously delivered to the driver, the control device 240 may determine to change only the volume of the music while maintaining the type of stimulus (music play). The control device 240 may transmit a control signal to the speaker 50 to play the music at a louder volume (for example, volume 15) than a volume (for example, volume 10) which has been used previously.

### Changing Autonomous Driving Level

In step S560, the control device 240 changes an autonomous driving level depending on the change of the alertness level and requests autonomous driving in accordance with the changed level from the autonomous driving control unit 230. For example, when the alertness level of the driver is increased, the control device 240 may request the autonomous driving control unit 230 to lower the autonomous driving level.

Next, the process returns to step S360 to transmit a stimulus with the type or property changed in step S530 or S540 to the driver and steps illustrated in FIG. 3 or 5 may be repeatedly performed until the driving ends.

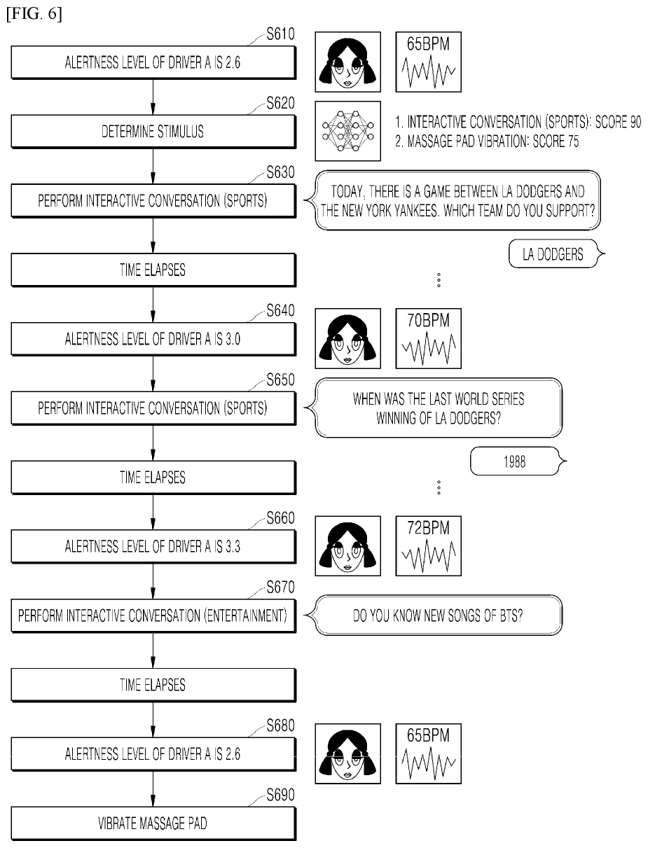

FIG. 6 is a view illustrating an exemplary scenario for improving an alertness of a driver according to an exemplary embodiment of the present disclosure.

Referring to FIG. 6, at first, the control device 240 determines the alertness level of the driver A as a level 2.6 based on information from the biometric sensor 13 and the image sensor 70 (S610).

When the alertness level of the driver A is 2.6, the alertness-stimulus learning engine 249 may output the interactive conversation (sports) as the most effective stimulus (score 90) and the vibration of the massage pad as a second effective stimulus (score 75) and the control device 240 determines the interactive conversation (sports) as a stimulus to be delivered to the driver (S620).

The IVR engine 243 performs the conversation on the sports with the driver through the speaker 50 and the acoustic sensor 70 (S630). For example, the IVR engine 243 asks a question that “There is a game between LA Dodgers and the New York Yankees, today. Which team do you support?” to induce an answer of the driver.

After a predetermined time (for example, one minute) has elapsed, the control device 240 determines the alertness level of the driver A after the interactive conversation as a level 3.0 based on information from the biometric sensor 13 and the image sensor 70 (S640).

Since the increased alertness level (3.0−2.6=0.4) exceeds the first threshold value (0.2) and the second threshold value (0.3), the conversation for the same subject (property) may be continued. For example, the IVR engine 243 asks a question that “When was the last World Series winning of the LA Dodgers?” to continue the conversation regarding the sports (S650).

After a predetermined time has elapsed, the control device 240 determines the alertness level of the driver A after the interactive conversation as a level 3.3 based on information from the biometric sensor 13 and the image sensor 70 (S660).

Since the increased alertness level (3.3−3.0=0.3) exceeds the first threshold value (0.2), but does not exceed the second threshold value (0.3), the subject (property) of the interactive conversation is changed. For example, the IVR engine 243 asks a question that “Do you know the new songs of BTS?” to perform conversation regarding entertainment (S670).

After a predetermined time has elapsed, the control device 240 determines the alertness level of the driver A after the interactive conversation as a level 2.6 based on information from the biometric sensor 13 and the image sensor 70 (S680).

Since the increased alertness level (2.6−3.3=−0.7) is lower than the first threshold value, the type of stimulus is changed. In the previous step S620, when the alertness level of the driver A is 2.6, the control device 240 determines the interactive conversation (sports) as a stimulus delivered to the driver. However, when the same stimulus is repeated, the improvement effect of the alertness by the stimulus is low as compared with the effect when the stimulus is applied at first. Therefore, at this time, the control device 240 determines the vibration of the massage pad which is determined as a second effective stimulus when the alertness level of the driver A is 2.6, as a stimulus to be delivered to the driver and outputs a control signal for operating the massage pad 120 (S690).

The example embodiments described above may be implemented through computer programs executable through various components on a computer, and such computer programs may be recorded in computer-readable media. Examples of the computer-readable media include, but are not limited to: magnetic media such as hard disks, floppy disks, and magnetic tape; optical media such as CD-ROM disks and DVD-ROM disks; magneto-optical media such as floptical disks; and hardware devices that are specially configured to store and execute program codes, such as ROM, RAM, and flash memory devices.

The computer programs may be those specially designed and constructed for the purposes of the present disclosure or they may be of the kind well known and available to those skilled in the computer software arts. Examples of program code include both machine code, such as produced by a compiler, and higher level code that may be executed by the computer using an interpreter.

As used in the present application (especially in the appended claims), the terms ‘a/an’ and ‘the’ include both singular and plural references, unless the context clearly states otherwise. Also, it should be understood that any numerical range recited herein is intended to include all sub-ranges subsumed therein (unless expressly indicated otherwise) and therefore, the disclosed numeral ranges include every individual value between the minimum and maximum values of the numeral ranges.

Also, the order of individual steps in process claims of the present disclosure does not imply that the steps must be performed in this order; rather, the steps may be performed in any suitable order, unless expressly indicated otherwise. In other words, the present disclosure is not necessarily limited to the order in which the individual steps are recited. All examples described herein or the terms indicative thereof (“for example”, etc.) used herein are merely to describe the present disclosure in greater detail. Therefore, it should be understood that the scope of the present disclosure is not limited to the example embodiments described above or by the use of such terms unless limited by the appended claims. Also, it should be apparent to those skilled in the art that various alterations, substitutions, and modifications may be made within the scope of the appended claims or equivalents thereof.

The present disclosure is thus not limited to the example embodiments described above, and rather intended to include the following appended claims, and all modifications, equivalents, and alternatives falling within the spirit and scope of the following claims.

What is claimed is:

1. A method for preventing drowsy driving, the method comprising:
determining a first alertness level of a driver based on monitoring information from at least one monitoring unit equipped in a vehicle;
determining a first stimulus corresponding to at least one of a plurality of available stimulation units, based on the determined first alertness level; and
operating at least one stimulation unit corresponding to the first stimulus.

2. The method according to claim 1, further comprising:
identifying an identity of the driver,
wherein determining a first stimulus comprises determining the first stimulus based on the identity of the driver and the first alertness level.

3. The method according to claim 1, further comprising:
after operating at least one stimulation unit, determining a second alertness level of the driver;
determining a second stimulus corresponding to at least one of the plurality of available stimulation units when a difference between the second alertness level and the first alertness level is equal to or lower than a first threshold value; and
operating at least one stimulation unit corresponding to the second stimulus,
wherein the second stimulus is of a different type from the first stimulus.

4. The method according to claim 1, further comprising:
after operating at least one stimulation unit, determining a second alertness level of the driver; and
changing a property of the first stimulus and operating the at least one stimulation unit corresponding to the first stimulus when the difference between the second alertness level and the first alertness level is equal to or lower than a second threshold value.

5. The method according to claim 4, wherein determining a first stimulus comprises determining interactive conversation as the first stimulus, and changing a property of the first stimulus comprises changing a subject of the interactive conversation.

6. The method according to claim 3, wherein determining a first stimulus comprises:
providing the first alertness level as input data to an artificial neural network which has been trained to output one or more stimuli selected in accordance with the alertness level of the driver; and
determining the first stimulus based on output data from the artificial neural network, and
the method further comprises:
providing the second alertness level or a difference between the second alertness level and the first alertness level together with information on the first stimulus, as training data, to the artificial neural network in order to train the artificial neural network.

7. The method according to claim 1, further comprising:
activating autonomous driving control of the vehicle when the first alertness level is equal to or lower than a threshold level.

8. A vehicle, comprising:
at least one monitoring unit to monitor a state of a driver;
a plurality of stimulation units to deliver a stimulus to the driver; and
a control device configured to:
determine a first alertness level of the driver based on monitoring information from the at least one monitoring unit;
determine a first stimulus corresponding to at least one of the plurality of stimulation units, based on the determined first alertness level; and
output a control signal to operate a first stimulation unit corresponding to the first stimulus.

9. The vehicle according to claim 8, wherein the control device is further configured to identify an identity of the driver and determine the first stimulus based on the identity and the first alertness level of the driver.

10. The vehicle according to claim 8, wherein the control device is further configured to:
determine a second alertness level of the driver after operating the first stimulation unit; and
output a control signal to operate a second stimulation unit which is different from the first stimulation unit when a difference between the second alertness level and the first alertness level is equal to or lower than a first threshold value.

11. The vehicle according to claim 10, wherein the control device is further configured to output the control signal to change a property of the stimulus to the first stimulus unit when the difference between the second alertness level and the first alertness level exceeds the first threshold value and is equal to or lower than a second threshold value.

12. The vehicle according to claim 11, wherein the control device further comprises an interactive voice response (IVR) engine configured to perform conversation with the driver using an artificial neural network which has been trained to analyze a meaning of a driver's voice and output a voice related to the analyzed meaning of the driver's voice, and
wherein the control device is further configured to:
determine an interactive conversation using the IVR engine as the first stimulus; and
change a subject of the interactive conversation when the difference between the second alertness level and the first alertness level exceeds the first threshold value and is equal to or lower than the second threshold value.

13. The vehicle according to claim 10, wherein the control device further comprises an alertness-stimulus learning engine comprising an artificial neural network which has been trained to output one or more stimuli selected in accordance with the alertness level of the driver, and
wherein the control device is further configured to:
determine the first stimulus based on output data from the alertness-stimulus learning engine; and
provide the second alertness level or the difference between the second alertness level and the first alertness level to the alertness-stimulus learning engine as training data to train the alertness-stimulus learning engine.

14. The vehicle according to claim 8, further comprising:
an autonomous driving control unit,
wherein the control device is further configured to output a control signal to operate the autonomous driving control unit when the first alertness level is equal to or lower than a threshold level.
